(19) United States
(12) Patent Application Publication
(10) Pub. No.: US 2008/0095796 A1
(43) Pub. Date: Apr. 24, 2008

(54) Novel Infectious Burasal Disease Virus And Vaccine Containing Said Virus

(75) Inventors: Kenichi Yamazaki, Kumamoto-ken (JP); Kozo Takase, Kagoshima-ken (JP); Hideyuki Ohta, Kumamoto-ken (JP); Masashi Sakaguchi, Kumamoto-ken (JP)

Correspondence Address: BROWDY AND NEIMARK, P.L.L.C.;624 NINTH STREET, NW, SUITE 300, WASHINGTON, DC, 20001-5303, US

(73) Assignee: JURIDICAL FOUNDATION THE CHEMOSERO-THERAPEUTIC RESEARCH INSTITUTE, KUMAMOTO-KEN (JP)

(21) Appl. No.: 11/547,160

(22) PCT Filed: Sep. 7, 2004
(86) PCT No.: PCT/JP04/12977

§ 371 Date: Oct. 2, 2006

(30) Foreign Application Priority Data
Mar. 31, 2004 (JP) 2004-106922

(51) Int. Cl. A61K 39/00; A61P 31/14; C07H 21/00; C07K 14/00; C12N 7/00

(52) U.S. Cl. 424/204.1; 435/235.1; 530/350; 536/23.72

(57) ABSTRACT

Novel infectious bursal disease (IBD) virus not protectable by the conventional IBD vaccines was isolated and a novel vaccine efficacious to said virus is provided. Novel IBD virus TY2 strain was isolated and its passage strains were obtained. TY2 strain has a distinct genetic type from those of the known IBD viral strains wherein a hypervariable region in VP2, a major host protective antigenic region of said virus, is cleaved with neither TaqI nor SspI but cleaved with MvaI into fragments of about 52 bp and about 422 bp. TY2 strain or the passage strain of said virus allows for production of an excellent vaccine to IBD.

### TECHNICAL FIELD

The present invention relates to a novel avian infectious bursal disease viral strain. More particularly, the present invention relates to a vaccine and a vaccine composition to infectious bursal disease utilizing said novel viral strain.

### BACKGROUND ART

Infectious Bursal Disease (hereinafter referred to as “IBD”) is an acute infectious disease of poultry such as chickens or turkey caused by IBD virus, which is a double-stranded RNA virus belonging to Birnaviridae. IBD virus is divided into two serotypes, i.e. serotype 1 and serotype 2 wherein serotype 1 is derived from and is pathogenic to chickens whereas serotype 2 is predominantly isolated from turkey and has extremely poor pathogenicity. A gene of IBD virus has a segment A of about 3.3 kb and a segment B of about 2.8 kb in length. The segment A codes for the protein of 110 kD: NH₂-VP2-VP4-VP3-COOH wherein VP2, a structural protein, is a major host protective antigen. Variance of a hypervariable region in VP2, a region corresponding to the amino acids No. 206 to No. 350 in VP2, may alter antigenicity or pathogenicity of IBD virus. On the other hands the segment B codes for VP1, a multifunctional protein (polymerase) of 90 kD.

Bursa of Fabricius (hereinafter referred to as “BF”) is one of the major lymphoid tissues in avian. In chickens, it reaches its maximum size at the age of about ten weeks and then undertakes retraction with sexual maturation. IBD virus exhibits strong affinity to Progenitor of B cells in the BF, induces immunosuppression by infection and may be the cause of opportunistic infection and a failure in vaccine immunization. A chicken of 3 to 12 weeks old has a high sensitivity to IBD virus and when infected with the virus develops symptoms such as depression of spirits, ruffling up of feather, gloom, diarrhea etc. and even dies in severe cases. Pathological observation shows swelling or atrophy, edema, yellowing or hemorrhage in the BF, atrophy in the thymus, hemorrhage in the skeletal muscle, swelling and formation of white spots in the spleen, swelling or fading in the liver, swelling or fading in the kidney, dilation of the urinary tubule, etc. Even in non-lethal cases, IBD may be accompanied by another infectious diseases and cause sometimes great economical damages.

In 1950s, IBD occurred for the first time in the Gumboro district, Delaware, U.S. In Japan, it was firstly reported during the first half of the decade of 1960s and now prevails all over the country. From the summer of 1990 onward, there was an outbreak of highly pathogenic IBD, characterized by extremely high lethality (about several % to about 60%) as compared to chickens infected with the conventional IBD virus, in various parts of the country that has caused enormous damages.

Since no efficacious remedy has been found for IBD, prevention by vaccination is the most important countermeasure. For vaccination as commonly used, an inactivated vaccine is administered to breeding chickens to enhance maternal antibodies in a chicken and then a live vaccine is administered to a chicken of about 2 to 4 weeks old to accelerate an antibody titer.

A variety of IBD vaccines has been reported up till the present including a vaccine comprising the structural proteins of IBD virus such as VP2, VP3 and VP4 proteins (see Patent reference 1), an inactivated vaccine of IBD virus IQ and 91-6 strains (see Patent reference 2), and a vaccine utilizing IBD virus YH-91-CLC strain (see Patent reference 3), and the like.

For the amino acids in a hypervariable region of strains attenuated and adapted through cell culture from highly pathogenic IBD virus, it is reported that variations may be predicted for the amino acid No. 279 from aspartic acid to asparagine and for the amino acid No. 284 from alanine to threonine (see Non-patent reference 1).

Although currently practicable live vaccines are efficacious even to highly pathogenic IBD, onset of IBD has recently been reported in spite of administration of these live vaccines in Japan.
Patent reference 1: Japanese patent publication No. 194597/1993
Patent reference 2: Japanese patent publication No. 67634/1995
Patent reference 3: Japanese patent publication No. 2001-86983
Non-patent reference 1: Yamaguchi, T. et al., Virology, 223, pp.219-223 (1996)

### DISCLOSURE OF THE INVENTION

### (Technical Problem to be Solved by the Invention)

Since variant viral strains may emerge in the field that have new properties not protectable by the conventional vaccines, there is a need to develop a vaccine with the use of novel viral strains as appropriate to such variant viral strains.

### (Means for Solving the Problems)

Under the circumstances, the present inventors have continued research assiduously and as a consequence have isolated from the field IBD virus TY2 strain with a low cross-reactivity with the conventional vaccines in view of antigenicity, studied its properties, and successfully produced a viral strain with a low lethal pathogenicity to a chicken by subculture of TY2 strain in the chorioallantoic membrane (hereinafter referred to as “CAM”) of embryonated eggs and/or in chick embryos and a viral strain adapted to chicken embryonic fibroblasts (hereinafter referred to as “CEFs”)

Thus, the present invention relates to (1) a novel IBD virus TY2 strain; (2) a passage strain of said viral strain; (3) an IBD vaccine comprising as an active ingredient IBD virus TY2 strain or a passage strain of said viral strain; (4) an IBD vaccine composition comprising as an active ingredient IBD virus TY2 strain or a passage strain of said viral strain and a pharmaceutically acceptable carrier; (5) a gene comprising a nucleotide sequence encoding the amino acid sequence as depicted in SEQ ID NO: 1; (6) a peptide comprising the amino acid sequence as depicted in SEQ ID NO: 1; (7) a gene comprising a nucleotide sequence encoding the amino acid sequence as depicted in SEQ ID NO: 3; (8) a peptide comprising the amino acid sequence as depicted in SEQ ID NO: 3; (9) a gene comprising a nucleotide sequence encoding the amino acid sequence as depicted in SEQ ID NO: 4 or 5; and (10) a peptide comprising the amino acid sequence as depicted in SEQ ID NO: 4 or 5.

### (More Efficacious Effects than Prior Art)

IBD virus TY2 strain or a passage strain of said viral strain allows for production of a vaccine that is efficacious to IBD variant viral strains that may have new properties not protectable by the conventional vaccines.

### BRIEF DESCRIPTION OF DRAWINGS

FIG. 1 shows an outline of a test schedule for isolation of IBD virus.

FIG. 2 is a photograph showing results of electrophoresis as gene cleavage patterns with TaqI for each of isolated IBD viruses and the known IBD viral strains. A to C show the gene cleavage patterns.

FIG. 3 is a photograph showing results of electrophoresis as gene cleavage patterns with SspI for each of isolated IBD viruses and the known IBD viral strains. D and E show the gene cleavage patterns.

FIG. 4 is a photograph showing results of electrophoresis as gene cleavage patterns with MvaI for each of isolated IBD viruses and the known IBD viral strains. F and G show the gene cleavage patterns.

FIG. 5 shows a transitional neutralizing antibody titer (geometric average) to IBD virus K strain with the time after in ovo vaccination of IBD virus TY2 strain.

FIG. 6 shows alignment of amino acid sequences of IBD virus TY2 strain and of various other IBD vaccine strains.

### BEST MODE FOR CARRYING OUT THE INVENTION

Novel IBD virus TY2 strain not protectable by the conventional vaccines may be isolated in the field as described in Example 1. In general, chickens reared in a farm usually have already received an IBD live vaccine and thus, when they are infected with a novel field strain of the virus that has a distinct antigenic region from that of the live vaccine, not only said novel strain but also the vaccine viral strain of said live vaccine, i.e. the virus having the conventional antigenic region, are likely to propagate together, which makes selective isolation of the novel strain difficult. In another approach, even if non- immunized SPF chickens not immunized with any IBD vaccine are reared together in a farm, they will simultaneously be infected with both the conventional virus and a novel viral strain to thereby make selective isolation of the novel strain difficult.

On the other hand, if SPF chickens immunized with the currently used IBD inactivated vaccine are reared together in a farm as a sentinel chicken, the field strain that has the overlapping antigenic region with that of the inactivated vaccine strain, i.e. the strain protectable by the conventional vaccine, could not propagate after entering the body of the sentinel chickens whereas those field strains that have a distinct antigenic region from that of the inactivated vaccine strain could propagate in the body of the sentinel chickens. Besides, since the sentinel chickens are immunized with the inactivated vaccine, propagation of the vaccine strain in the body of the chickens will not occur unlike chickens administered with the live vaccine and hence the approach is suited for isolation of a field viral strain having a distinct antigenic region from that of the conventional vaccines. Accordingly, this approach may be used for obtaining novel IBD viral strain not protectable by the conventional vaccines.

A genetic type of IBD virus TY2 strain obtained in accordance with the present invention was compared with those of the known IBD viral strains by restriction fragment length polymorphism (RFLP) using three restriction enzymes TaqI, SspI and MvaI to prove that TY2 strain exhibited a distinct cleavage patter from those of the known IBD viral strains wherein TY2 strain was cleaved with neither TaqI nor SspI but cleaved with MvaI into fragments of about 52 bp and about 422 bp (Example 1). Thus, it was demonstrated that IBD virus TY2 strain obtained in accordance with the present invention had a definitely distinct genetic type from those of the known IBD viral strains.

SEQ ID NO: 3 shows the amino acid sequence of a hypervariable region in VP2, corresponding to the sequence of the amino acids No. 206 to No. 350, VP2 being a major host protective antigenic region of IBD virus TY2 strain. SEQ ID NOs: 1 and 2 show the amino acid sequence of VP2 of said TY2 strain and the nucleotide sequence encoding said amino acid sequence, respectively.

Homology analysis for the amino-acid sequence of a hypervariable region in VP2 of IBD virus TY2 strain obtained in accordance with the present invention was performed with those of the known IBD viral strains [K strain (SEQ ID NO: 6), 228E strain (SEQ ID NO: 7), 2512 strain (SEQ ID NO: 8), Bursine2 (Lukert BP) strain (SEQ ID NO: 9), D78 strain (SEQ ID NO: 10) and V877 strain (SEQ ID NO: 11)] to demonstrate that the hypervariable region exhibited a maximum homology of 92.4% and a minimum homology of 89.7%.

IBD virus TY2 strain obtained in accordance with the present invention was also studied for its pathogenicity to demonstrate that no abnormality in clinical symptoms was observed as described in Example 2, proving low pathogenicity of the strain. However, it was revealed by macroscopic observation with autopsy after administration of TY2 strain with lapse of time that the strain had a strong pathogenicity to the BF since atrophy of the BF was observed at Day 3 onward, a weight ratio of the BF to the body weight was 0.2% or less at Day 4 onward with much severity, and gelatinous degeneration of the BF was observed at Day 3 to Day 5 (Example 2).

For cross-reactivity of IBD virus TY2 strain obtained in accordance with the present invention with other IBD viral strains, it was found that TY2 strain had a low cross-reactivity at least to K strain (Example 3).

IBD virus TY2 strain obtained in accordance with the present invention, when administered in ovo to SPF embryonated eggs, exhibited an enhanced neutralizing antibody titer to the known IBD viral strains to thereby prove its excellent protection from highly pathogenic IBD virus (Example 4).

Said TY2 strain was subcultured to obtain an attenuated passage strain. When said passage strain was administered to SPF chickens, no lesion in the BF was observed to reveal that the passage strain had a reduced pathogenicity to the BF (Example 8). Said passage strain is also characterized by that the amino acid No. 284 (alanine) in the amino acid sequence of VP2 is replaced with threonine. Besides, it was confirmed that said passage strain had an ability to produce an antibody to IBD virus K strain to thereby reveal its immunogenicity to the conventional IBD viruses, to which the passage strain is likely to have a low cross-reactivity (Example 9).

By way of an example, said passage strain includes IBD virus TY2-CEF1 and TY2-CEF2 strains as adapted to CEFs wherein the amino acid sequences of a hypervariable region in VP2 of these TY2-CEF strains are indicated in SEQ ID NOs: 4 and 5, respectively.

Analysis of a gene sequence of IBD virus TY2 strain and the like in accordance with the present invention may be carried out by the known methods, e.g. as described in Molecular Cloning; A Laboratory Manual, Cold Spring Harbor Laboratory, Cold Spring Harbor, N.Y. (2001).

A vaccine as used herein refers to an inactivated vaccine obtained by culture of IBD virus TY2 strain or IBD viral strains having equivalent antigenicity to that of TY2 strain followed by inactivation, or a live vaccine utilizing attenuated live viruses per se. A live vaccine viral strain may be produced by the know methods such as subculture (Lukert, P. D. et al., Disease of Poultry, 9th ed., Iowa State Univ. Press, 690-699, 1991), e.g. CAM passage with embryonated eggs or CEF passage for attenuation followed by plaque cloning etc. A live vaccine is such that said attenuated strain is cultured with embryonated eggs or L culture cells such as CEFs. An inactivated vaccine is such that the strain is inactivated by the known methods such as physical treatment including e.g. heating, radiation of ultraviolet ray, and chemical treatment with e.g. formalin, beta-propiolactone etc. It is also possible to construct a DNA vaccine or a vector vaccine by employing VP2 sequencing of TY2 strain or its passage strain (Tsukamoto, K., et al., Virology, 257, 352-362, 1999 and Sonoda, K., et al., J. of Virology, 74(7), 3217-26, 2000).

A route of administration of the vaccine in accordance with the present invention includes oral, eye drop, nasal drop, intramuscular, intravenous, subcutaneous, or in ovo administration. For administration of an inactivated vaccine, intramuscular, intraperitoneal or subcutaneous administration is preferable wherein immunizing effect may be enhanced by co-administration of the known adjuvants such as vegetable oils, e.g. sesame oil, rape oil, etc., mineral oils, e.g. light liquid paraffin, etc., aluminum hydroxide gel, aluminum phosphate gel, and the like.

An animal to be applied with the IBD vaccine in accordance with the present invention includes poultry such as chickens, turkey, quail, duck, etc. The vaccine may be administered at any time. In case of chickens for meat such as broiler, the live vaccine may preferably be administered to a chicken of 2 to 4 weeks old when maternal antibodies may disappear. In case of chickens for eggs or breeding chickens, the live vaccine may be administered at 2 to 4 weeks old and the live or inactivated vaccine may be administered at 70 days old or thereabouts. For the inactivated vaccine in an oil adjuvant formulation, it may be administered at 80 days old or thereabouts whereas for the inactivated vaccine in other formulation than oil, e.g. in an aluminum hydroxide gel formulation, it may preferably be administered twice at 80 days old or thereabouts and at 110 days old or thereabouts.

Culture of IBD virus TY2 strain in accordance with the present invention and/or its passage strain may not be restricted and may be carried out with the known methods. An example of the culture process is described below. A viral solution (1.0 mL) of a passage strain of IBD virus TY2 strain, prepared with a maintenance medium or PBS to a fixed amount of the virus (e.g. 10^{4.0} TCID₅₀/mL), is inoculated to CEFs (in a cell culture bottle with an area: 25 cm²) previously cultured at 37° C. for 24 to 48 hours in the presence of 5% C0₂ in a routine manner. Then, the cells are cultured at 37° C. for 24 to 96 hours in the presence of 5% C0₂ and all the culture supernatant is recovered and centrifuged (3000 rpm, 20 minutes) at 4° C. to collect a supernatant.

IBD virus TY2 strain and a passage strain of said virus in accordance with the present invention, which may be stored at −80° C. or less for a long period of time, is stored in Juridical Foundation The Chemo-Sero-Therapeutic Research Institute and may be distributed at request.

### EXAMPLE

The present invention is explained in more detail by means of the following Examples but should not be construed to be limited thereto.

### Example 1

### Isolation of IBD virus from commercial broiler farm

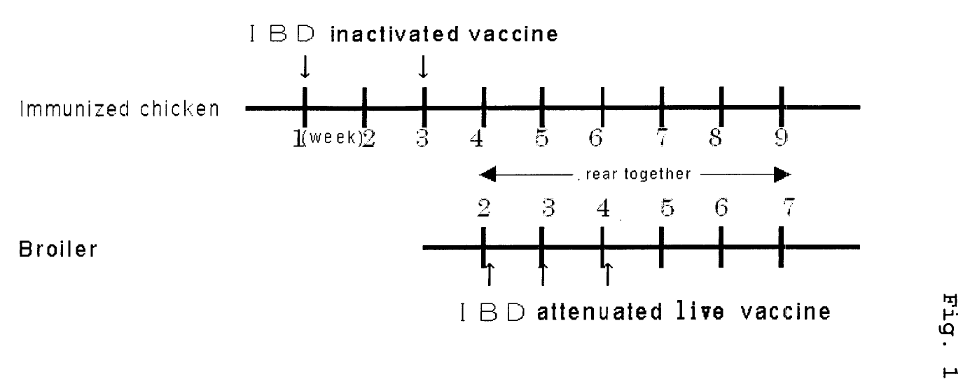

Highly immunized specific pathogen free (hereinafter referred to as “SPF”) chickens of 4 weeks old (immunized SPF chickens), obtained by immunizing in laboratory SPF chickens at 1 and 3 weeks old with IBD inactivated vaccine (Nisseiken IBD inactivated vaccine, Nisseiken Co., Ltd.) utilizing the conventional IBD viral strain, and untreated SPF chickens of 4 weeks old were reared together with broilers of 2 weeks old of a commercial broiler farm for 5 weeks. In the broiler farm, broilers received the commercially available IBD attenuated live vaccine (IBD live vaccine “Kaketsuken”, Juridical Foundation The Chemo-Sero-Therapeutic Research Institute) at 2, 3 and 4 weeks old by mixing the vaccine in feeding water (FIG. 1).

After starting rearing the immunized SPF chickens, the SPF chickens with no treatment and the broilers together, each 5 chickens from the respective-group were brought into a laboratory for autopsy and the BFs were taken. The BFs were pooled for each group and used for preparing 10% emulsion of the BF. After amplification by reverse transcriptase polymerase chain reaction (RT-PCR) and polymerase chain reaction (PCR), a genetic type was studied by RFLP using three restriction enzymes TaqI, SspI and MvaI. Extraction of the viral nucleic acids was performed with QIAamp Viral RNA Mini Kit (QIAGEN). A solution (60 μL) of the extracted RNAs was prepared from the emulsion (140 uL) of the BF. Using this solution of the viral extracted RNAs as a template, RT-PCR was performed for a hypervariable region of VP2 with OneStep RT-PCR Kit (QIAGEN) using primers as indicated in SEQ ID NOs: 12 and 13, P2.3 (5′-CCCAGAGTCTACACCATA-3′) and RP5.3 (5′-TCCTGTTGCCACTCTTTC-3′).

An RT reaction solution as depicted in Table 1 was prepared. RT reaction was performed at 50° C. for 30 minutes and then, after inactivating a reverse transcriptase by heating at 94° C. for 4 minutes, 25 cycles of heat denaturation at 94° C. for 1 minute, annealing at 52° C. for 1 minute and elongation at 72° C. for 2 minutes were carried out, followed by elongation at 72° C. for 10 minutes.

TABLE 1

| | | |
|---|---|---|
| Primer (forward) | 1 | μL |
| Primer (reverse) | 1 | μL |
| dNTP mixture | 2 | μL |
| RT-PCR Enzyme | 2 | μL |
| 5 × RT-PCR | 10 | μL |
| 5 × Q-Solution | 10 | μL |
| Rnase-free water | 23 | μL |
| RNA extract | 1 | μL |
| Total | 50 | μL |

For the obtained gene fragments, PCR was further performed using the same primers so as to ensure a sufficient amount of the gene fragments for RFLP analysis. A reaction solution as depicted in Table 2 was prepared. After heating at 94° C. for 4 minutes, 25 cycles of heat denaturation at 94° C. for 1 minute, annealing at 52° C. for 1 minute and elongation at 72° C. for 2 minutes were carried out, followed by elongation at 72° C. for 10 minutes. Electrophoresis with 1.5% agarose gel confirmed that the desired DNA fragments were obtained as PCR products. For RFLP analysis, this DNA fragment (1 μL) was reacted with either a reaction solution prepared with TaqI (1 μL), 10×H buffer (2 μL) and sterilized distilled water (16 μL) for 65° C. for 60 minutes, a reaction solution prepared with SspI (1 μL), 10×I buffer (2 μL) and sterilized distilled water (16 μL) for 37° C. for 60 minutes, or a reaction solution prepared with MvaI (1 μL), 10×K buffer (2 μL) and sterilized distilled water (16 μL) for 37° C. for 60 minutes, respectively. After reaction, electrophoresis was performed with PAGEL NPG-1020L (ATTO CORPORATION) to analyze the cleaved DNA fragments.

TABLE 2

| | | |
|---|---|---|
| Primer (forward) | 1 | μL |
| Primer (reverse) | 1 | μL |
| Master mixture | 25 | μL |
| H₂O | 22 | μL |
| RT-PCR solution | 1 | μL |
| Total | 50 | μL |

As a result, patterns A to G as described below were observed.
Pattern A: No cleavage with TaqI
Pattern B: Cleavage with TaqI to produce fragments of about 223 bp and about 251 bp
Pattern C: Cleavage with TaqI to produce fragments of about 100 bp and about 374 bp
Pattern D: No cleavage with SspI
Pattern E: Cleavage with SspI to produce fragments of about 201 bp and about 273 bp
Pattern F: Cleavage with MvaI to produce fragments of about 52, 59, 70, 139 and 154 bp
Pattern G: Cleavage with MvaI to produce fragments of about 52 and about 422 bp

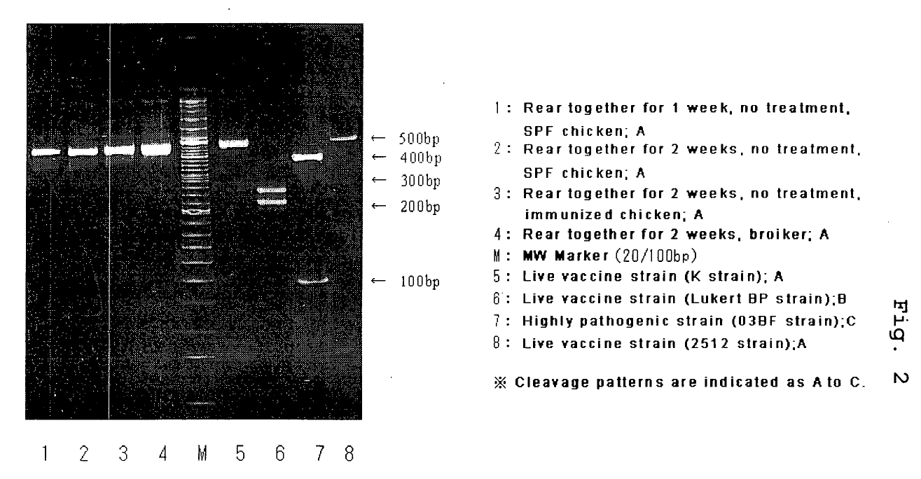

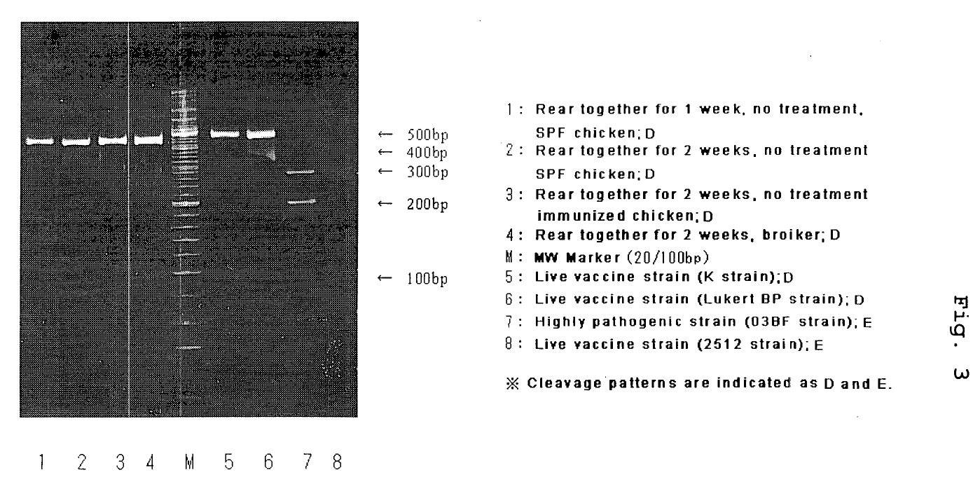

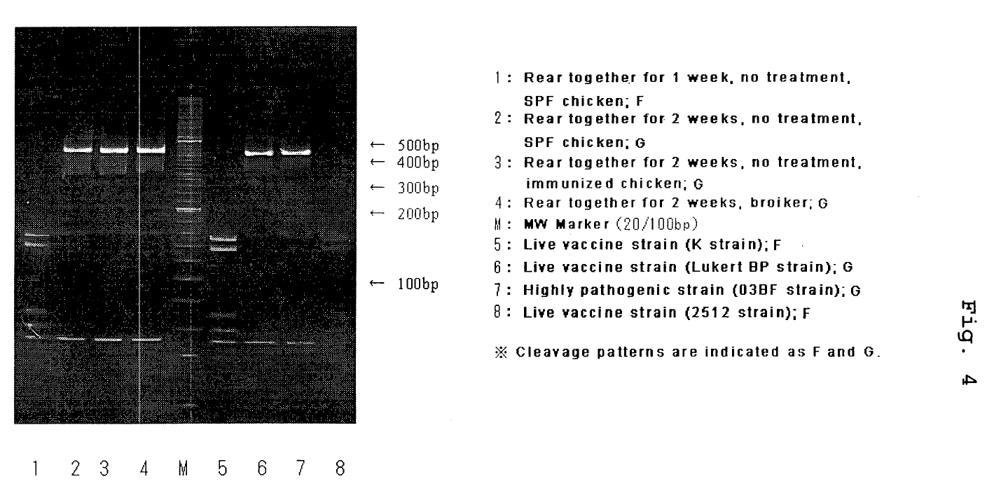

Excepting SPF chickens with no treatment at Week 1 after rearing together wherein the same genetic type (gene cleavage pattern I: A, D, F) as that of IBD attenuated live vaccine as used in the farm was detected, a distinct genetic type (gene cleavage pattern II: A, D, G) from the conventional attenuated vaccine or highly pathogenic strain was detected at Weeks 2 and 3 after rearing together for immunized SPF chickens and SPF chickens with no treatment and at Weeks 2 to 4 after rearing together for broilers (FIGS. 2 to 4, Tables 3 and 4).

TABLE 3

| | Isolated strain | | | | | | | |
|---|---|---|---|---|---|---|---|---|
| Restric- | Not | Not | Immu- | | Vaccine strain | | | |
| tion | treated | treated | nized | Broilers | | Lukert | O3 | 2512- |
| enzyme | (1)¹⁾ | (2) | (2) | (2) | K | BP | BF | G61 |
| TaqI | A | A | A | A | A | B | C | A |
| SspI | D | D | D | D | D | D | E | E |
| MvaI | F | G | G | G | F | G | G | F |
| pattern | I | II | II | II | I | III | IV | V |

¹⁾No. of weeks after rearing together

TABLE 4

Homology of genetic type

| | 4¹⁾* | 5* | 6* | 7 | 8 | 9 |
|---|---|---|---|---|---|---|
| Broilers | — | — | II | II | II | — |
| Immunized | — | — | II | II | — | — |
| Not | — | I | II | II | — | — |
| treated | | | | | | |

¹⁾Age of weeks of SPF chickens
*IBD attenuated live vaccine (K strain; gene cleavage type of I) was administered to every groups.

### Example 2

### Pathogenicity of IBD virus TY2 strain to SPF chickens

A 10% emulsion of the BF from immunized SPF chickens at Week 2 after rearing together in Example 1 was administered orally to SPF chickens of 5 weeks old for subculture for two filial generations in the BF. The obtained strain, called IBD virus TY2 strain, was studied for its pathogenicity to SPF chickens. IBD virus TY2 strain (104.1 EID₅₀/mL) was diluted 10-fold with PBS and 1.0 mL was orally administered to 40 SPF chickens of 5 weeks old. At Days 3, 4, 5, 7, 10 and 14 after administration, each 6 chickens were conducted autopsy and the lesion in the BF was observed with lapse of time. As a control with no treatment, 36 SPF chickens were prepared and each 6 chickens were similarly conducted autopsy before administration and with the same schedule as that of TY2 strain excepting at Day 4 after administration. The BFs were weighed and a weight ratio to the body weight was calculated. For preparing a tissue slice, the BFs were fixed in 10% formalin and HE-stained for histopathological observation.

As a result, no abnormality in clinical symptoms was observed after administration of TY2 strain. Macroscopic observation with autopsy after administration of TY2 strain with lapse of time proved atrophy in the BF at Day 3 and 0.2% or less of a weight ratio of the BF to the body weight at Day 4 onward with much severity. At Day 3 to Day 5, gelatinous degeneration in the BF was observed (Table 5). Observation with autopsy other than the BF demonstrated swelling and formation of white spots in the spleen at Day 3 to Day 10 and atrophy in the thymus at Day 4 to Day 10.

In histopathological observation of the BF, moderate reduction or disappearance of lymphocytes and severe shrinkage of the lymphoid follicles at Day 3 onward were seen. Slight infiltration of inflammatory cells, in particular, heterophils, in the lymphoid follicles was observed and edema in the interstitial region. At Day 7, there were observed necrosis of lymphocytes, in the lymphoid follicles, phagocytosis by macrophages and prominent net eye by the reticulum cells, and in the cortex region, thinning through reduction of lymphocytes and growth of the connective tissue. On the other hand, at Day 14, although the follicles were likely to recover since reconstitution of the follicular structure and appearance of activated follicles were observed, there were scarce follicles completely recovered, suggesting that it takes time for complete recovery.

TABLE 5

| | TY2 | | | | | | | | Control | |
|---|---|---|---|---|---|---|---|---|---|---|
| | | BF | | | | | | | | BF |
| | | Ratio | | | | | | | | Ratio |
| | Mean | to | | | | | | | Mean | to |
| | weight | B.W. | | | | Spleen | | Thymus | weight | B.W. |
| Day | (g) | (%) | *1 | *2 | *3 | *4 | *5 | *1 | (g) | (%) |
| Before | 230.0 | 0.45 | 0/6 | 0/6 | 0/6 | 0/6 | 0/6 | 0/6 | 230.0 | 0.45 |
| adm. | | | | | | | | | | |
| 3 | 275.0 | 0.27 | 6/6 | 6/6 | 0/6 | 3/6 | 3/6 | 0/6 | 306.7 | 0.37 |
| 4 | 310.0 | 0.20 | 10/10 | 10/10 | 0/10 | 2/10 | 7/10 | 4/10 | ND¹⁾ | ND |
| 5 | 291.7 | 0.17 | 6/6 | 6/6 | 0/6 | 3/6 | 1/6 | 3/6 | 346.7 | 0.46 |
| 7 | 334.2 | 0.17 | 6/6 | 0/6 | 0/6 | 3/6 | 2/6 | 5/6 | 375.8 | 0.37 |
| 10 | 405.0 | 0.15 | 6/6 | 0/6 | 0/6 | 0/6 | 2/6 | 2/6 | 386.7 | 0.42 |
| 14 | 430.8 | 0.11 | 6/6 | 0/6 | 0/6 | 0/6 | 0/6 | 0/6 | 448.3 | 0.41 |

*1: Atrophy
*2: Gelatinous degeneration
*3: Hemorrhage
*4: Swelling
*5: Formation of white spots
¹⁾Not done

### Example 3

### Cross-reactivity of IBD virus TY2 strain with other IBD viral strains

In ovo cross-reactivity neutralization test was carried out to investigate cross-reactivity of IBD virus TY2 strain with K strain (attenuated live vaccine strain), the conventional IBD virus. Each 0.6 mL of 5-fold dilution with PBS of antiserum to TY2 strain, antiserum to K strain and serum from SPF chickens as a control, and each 0.6 mL of serial dilutions by 10-fold from 10-1 to 10-4 of IBD virus TY2 strain and K strain, were mixed together for reaction at 37° C. for 1 hour (neutralization reaction) Each 0.2 mL of the mixture after neutralization reaction was inoculated to CAM of 5 SPF embryonated eggs of 11 days old. A week after the inoculation, the eggs were examined for their survival and, as for living eggs, the presence of viral propagation was detected by observing the liver of embryos after opening the eggs. Neutralization index for each viral strain was calculated from titers of SPF chickens antiserum and of each viral strain (Table 6).

As a result, a low cross-reactivity between TY2 strain and K strain was suggested.

TABLE 6

Antiserum

| | | K strain | TY2 strain |
|---|---|---|---|
| Antigen | K strain (4.5^{a)}) | 2.4^{b)} | 1.6 |
| | TY2 strain (5.7) | 1.6 | 3.0 |

^{a)}Viral content obtained by reaction with SPF control serum (Log₂EID₅₀/mL)
^{b)}Neutralization index (Log₂EID₅₀/mL)

### Example 4

### Immunogenicity of IBD virus TY2 strain through in ovo administration, and protective capacity against pathogenic IBD virus

IBD virus TY2 strain was subcultured in the BF of SPF chickens of 4 weeks old for 1 filial generation and then by inoculation to CAM of SPF embryonated eggs of 11 days old for 4 filial generations. The obtained passage strain was in ovo inoculated 10^{4.0}EID₅₀/0.05 mL/egg to embryonated eggs of 19 days old from chickens (layers) with positive maternal antibodies (test group). As a control, embryonated eggs with no inoculation were prepared. After hatching, each 8 chicks from the groups were conducted autopsy every week till Week 10 to determine a neutralizing antibody titer to IBD virus K strain. Besides, in order to investigate protection from highly pathogenic IBD virus, each 10 chickens were challenged at Weeks 6 and 9 by highly pathogenic IBD. virus K539 strain. Survival of the chickens up till four days after challenge was examined and, for living chickens, the protective capacity was evaluated by observing the autopsy finding of BF.

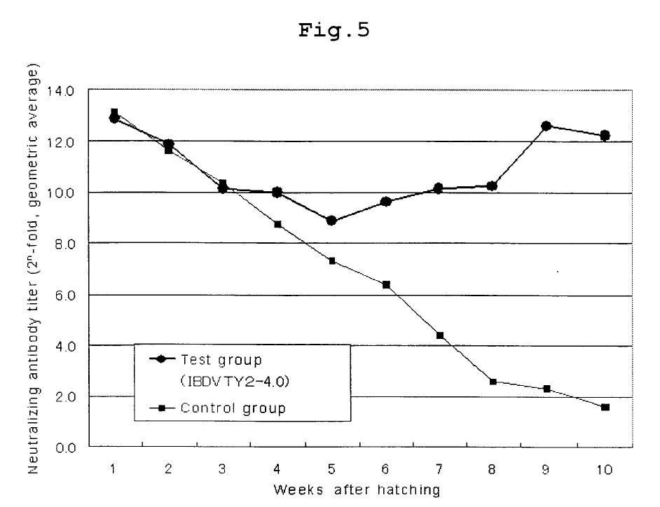

As a result, it was found that both groups kept high maternal antibodies immediately after hatching and their antibody titer declined with lapse of time. However, in the test group with inoculation of TY2 strain, increase in a neutralizing antibody titer was observed at Week 5 onward and the high titer was maintained up till Week 10 (FIG. 5). All the chickens in the test group also protected from the challenge of IBD virus K539 strain at Weeks 6 and 9 to thereby prove excellent protection from highly pathogenic IBD virus (Table 7).

TABLE 7

Week at challenge

| Group | 6 | 9 |
|---|---|---|
| Test group | 10/10^{a)} | 10/10 |
| Control | 0/10 | 0/10 |

^{a)}Presence of protection against challenge (No. of chickens protected/No. of chickens challenged)

### Example 5

### Determination of nucleotide sequence

Among the genes of IBD virus TY2 strain, VP2, the structural protein encoded by a segment A, was determined for its nucleotide sequence (SEQ ID NO: 2) by entrusting the determination to Hitachi Keisokuki Service K.K. (Ibaraki-ken, Tsukuba-shi) or TAKARA BIO Inc., Dragon Genomics Center (Mie-ken, Yokkaichi-shi).

### Example 6

### Homology search of IBD virus TY2 strain with other IBD vaccine strains

For homology analysis, a portion of VP2 region of IBD virus, the amino acid sequence of the amino acids No. 206 to No. 350 (hypervariable region), was used. For the known IBD vaccine strains, K strain, 22BE strain, 2512 strain, Bursine2 strain, D78 strain and V877 strain were used. The nucleotide sequences of each strain were obtained from the accession numbers in GSDB, DDBJ, EMBL and NCBI database [K strain was entrusted to TAKARA BIO Inc., Dragon Genomics Center for determination of the sequence. 228E strain: AAM90792, 2512 strain: AAG52760, Bursine2 strain: AAM21064, D78 strain: CAA75184 and V877 strain: CAE52969]. Homology analysis was performed for IBD virus TY2 strain with the known IBD vaccine strains using a genetic information processing software GENETYX-WIN (commercial name: manufactured by Software Development Co., Ltd.) and a nucleotide sequence automatic compilation software GENETYX-WIN/ATSQ (commercial name: manufactured by Software Development Co., Ltd.).

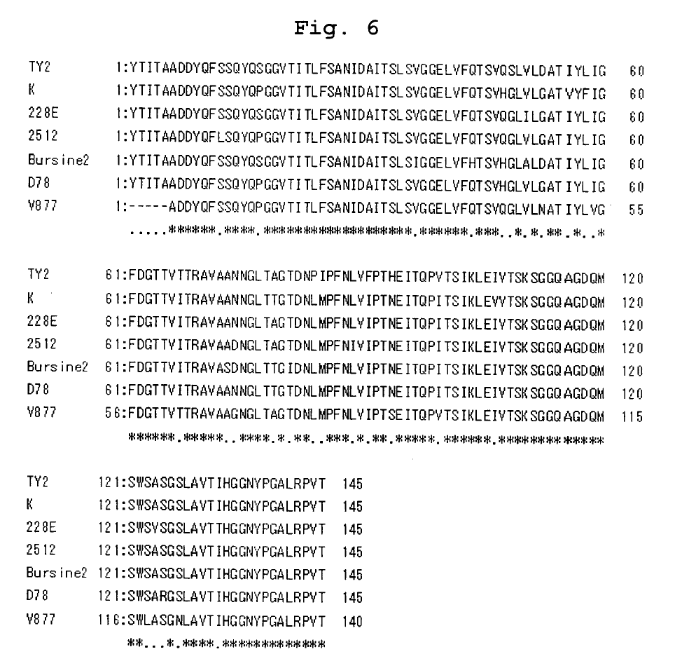

As a result, it was found that homology of TY2 strain with the conventional vaccine strains was at most 92.4% as shown in Table 8. Alignment of the amino acid sequence of IBD virus TY2 strain with those of the other IBD vaccine strains is shown in FIG. 6 wherein “*” shows that the amino acids from all the compared sequences conform and “.” depicts difference or absence of the amino acid in any of the sequences.

TABLE 8

| | Amino acid homology (%) | | | | | |
|---|---|---|---|---|---|---|
| Strain | K | 228E | 2512 | Bursine2 | D78 | V877 |
| TY2 | 90.3 | 92.4 | 91.7 | 89.7 | 91.7 | 92.1* |

*For V877 strain, alignment was made for the amino acids No. 211 to No. 350.

### Example 7

### Production of IBD virus TY2-CEF1 and TY2-CEF2 strains

IBD virus TY2 strain was inoculated to CAM of SPF embryonated eggs of 11 days old and, using a 20% emulsion of CAM and/or chicken embryo obtained three to four days after the inoculation, subculture was continued for 20 filial generations. The resulting passage strain of 20 filial generations was inoculated within the yolk sack of SPF embryonated eggs of 8 days old and then CEFs were cultured with the chicken embryo three days after the inoculation. The culture was centrifuged and the supernatant was inoculated to CEFs. Lyophilization and thawing was once carried out five to seven days after the inoculation and, using the supernatant from centrifugation, subculture was repeated for 8 filial generations. From subculture of the 3rd filial generation onward, a cytopathic effect could be seen to prove adaptation to CEFs. This passage strain of the 3rd filial generation from CEFs was further subcultured with CEFs for 3 filial generations by limiting dilution to clone “TY2-CEF1”.

Separately, IBD virus TY2 strain was orally administered to SPF chickens of about 5 weeks old to subculture in the BF for 1 filial generation. The resulting strain was inoculated to CAM of SPF embryonated eggs of 11 days old and, using a 20% emulsion of CAM and/or chicken embryo obtained three to four days after the inoculation, subculture was continued for 20 filial generations. The resulting passage strain of 20 filial generations was inoculated to CEFs. Lyophilization and thawing was once carried out five to seven days after the inoculation and, using the supernatant from centrifugation, subculture was repeated for 10 filial generations. From subculture of the 9th filial generation onward, a cytopathic effect could be seen to prove adaptation to CEFs. This passage strain of the 10th filial generation in CEFs was further subcultured with CEFs for 3 filial generations by limiting dilution to clone “TY2-CEF2”.

The amino acid sequence of VP2 hypervariable region of these two strains was analyzed to demonstrate that the amino acid No. 279 (corresponding to the amino acid No. 74 in SEQ ID NOs: 4 and 5) was asparagine and the amino acid No. 284 (corresponding to the amino acid No. 79 in SEQ ID NOs: 4 and 5) was mutated from alanine to threonine, which conformed with one of indexes for adaptation to CEFs as reported by T. Yamaguchi. et al., Virology, 223, 219-223, 1996.

### Example 8

### Pathogenicity of IBD virus TY2-CEF1 and TY2-CEF2 strains to SPF chickens

Each 10⁴TCID₅₀/mL of IBD virus TY2-CEF1 and TY2-CEF2 strains was orally administered to SPF chickens of about 4 weeks old. At Days 4 and 14 after administration, each 8 chickens, with exception for TY2-CEF1 strain at Day 14 of 9 chickens, were conducted autopsy and the lesion in the BF was observed. As a control with no treatment, 18 SPF chickens were prepared and each 6 chickens were similarly conducted autopsy before administration and at Days 4 and 14. The BFs were weighed and a weight ratio to the body weight was calculated.

The results are shown in Table 9. No abnormality in clinical symptoms was observed after administration of IBD virus TY2-CEF1 and TY2-CEF2 strains. Macroscopic observation of the BF with autopsy detected only slight atrophy in 1 among 8 chickens in the TY2-CEF1 group at Day 4 and sli-ght-to-moderate atrophy in 1 among 9 chickens in the TY2-CEF1 group and in 5 among 8 chickens in the TY2-CEF2 group at Day 14. These observations prove apparently reduced pathogenicity to the BF as compared to that of TY2 strain as described in Example 2.

TABLE 9

| | TY2-CEF1 | | | | TY2-CEF2 | | | | Control | | | |
|---|---|---|---|---|---|---|---|---|---|---|---|---|
| | | BF | | | | BF | | | | BF | | |
| | | Ratio | | | | Ratio | | | | Ratio | | |
| | Mean | to | | | Mean | to | | | Mean | to | | |
| | weight | B.W. | | | weight | B.W. | | | weight | B.W. | | |
| Day | (g) | (%) | *1 | *2 | (g) | (%) | *1 | *2 | (g) | (%) | *1 | *2 |
| Before | — | — | — | — | — | — | — | — | 159 | 0.44 | 0/6 | 0/6 |
| adm. | | | | | | | | | | | | |
| 4 | 235 | 0.35 | 1/8 | 0/8 | 239 | 0.41 | 0/8 | 0/8 | 257 | 0.32 | 0/6 | 0/6 |
| 14 | 380 | 0.34 | 1/9 | 0/9 | 409 | 0.26 | 5/8 | 0/8 | 385 | 0.44 | 0/6 | 0/6 |

*1: Atrophy
*2: Gelatinous degeneration

### Example 9

Ability to produce antibody of IBD virus TY2-CEF1 and TY2-CEF2 strains to SPF chickens In order to investigate an ability to produce an antibody to the conventional IBD virus, a neutralizing antibody titer to IBD virus K strain was measured using the chicken serum at Day 14 after administration as used in Example 8.

Each 0.05 mL of a maintenance medium was delivered to 96-well plate. To the 1st well was added 0.05 mL of an inactivated test serum at 56° C. for 30 minutes and diluted by 2-fold to the 12th well. IBD virus K strain (virus for neutralization) was diluted with a maintenance medium to 200 TCID₅₀/0.05 mL and each 0.05 mL of the virus for neutralization was added to the wells for reaction at 37° C. for 1 hour (neutralization reaction). Each 0.05 mL of CEFs prepared at 10⁶ cells/mL was added to the plate and cultured for 1 week. After completion of culture, the presence of a cytopathic effect was detected and a maximum fold of dilution at which no cytopathic effect was observed was reckoned as a neutralizing antibody titer of test serum (Table 10).

As a result, as shown in Table 10, a geometrical mean value of each titer was found to be 299-fold for IBD virus TY2-CEF1 strain and 235-fold for TY2-CEF2 strain to prove that both strains had an ability to produce an antibody to IBD virus K strain and hence immunogenicity to the conventional IBD virus which is likely to have a low cross-reactivity.

TABLE 10

| | Neutralizing Antibody titer |
|---|---|
| | (Fold, GM value) |
| TY2-CEF1 strain | 299 |
| TY2-CEF2 strain | 235 |
| Control | 1 |

1. Infectious bursal disease virus TY2 strain.

2. The infectious bursal disease virus TY2 strain of claim 1 wherein the gene coding for a hypervariable region in VP2, a major host protective antigenic region of said virus, is cleaved with neither TaqI nor SspI but cleaved with MvaI into fragments of about 52 bp and about 422 bp.

3. The infectious bursal disease virus TY2 strain of claim 1 or 2 wherein said hypervariable region has the amino acid sequence as indicated in SEQ ID NO: 3.

4. The infectious bursal disease virus TY2 strain of claim 1 wherein said VP2 has the amino acid sequence as indicated in SEQ ID NO: 1.

5. A passage strain from the infectious bursal disease virus TY2 strain of claim 1.

6. The passage strain of claim 1 wherein the amino acid No. 284, alanine, in the amino acid sequence of VP2 of said passage strain is replaced with threonine.

7. The passage strain of claim 1 wherein the hypervariable region within VP2 of said passage strain has the amino acid sequence as indicated in SEQ ID NO: 4 or 5.

8. The passage strain of claim 1 wherein said passage strain is a strain adapted to chicken embryonic fibroblasts (CEFs).

9. The passage strain of claim 1 wherein said passage strain is TY2 -CEF1 or TY2-CEF2 strain.

10. An infectious bursal disease vaccine comprising as an active ingredient the infectious bursal disease virus TY2 strain of claim 1.

11. An infectious bursal disease vaccine composition comprising as an active ingredient the infectious bursal disease virus TY2 strain of claim 1.

12. A gene from the infectious bursal disease virus TY2 strain of claim 1 comprising a nucleotide sequence coding for the amino acid sequence as indicated in SEQ ID NO: 3.

13. A hypervariable region peptide in VP2 of infectious bursal disease virus TY2 strain having the amino acid sequence as indicated in SEQ ID NO: 3.

14. A gene from the infectious bursal disease virus TY2 strain of claim 1 comprising a nucleotide sequence coding for the amino acid sequence as indicated in SEQ ID NO: 1.

15. A hypervariable region peptide in VP2 of infectious bursal disease virus TY2 strain having the amino acid sequence as indicated in SEQ ID NO: 1.

16. A gene from the passage strain from the infectious bursal disease virus TY2 strain of claim 1 comprising a nucleotide sequence coding for the amino acid sequence as indicated in SEQ ID NO: 4.

17. A hypervariable region peptide in VP2 of a passage strain from infectious bursal disease virus TY2 strain having the amino acid sequence as indicated in SEQ ID NO: 4.

18. A gene from the passage strain from the infectious bursal disease virus TY2 strain of claim 1 comprising a nucleotide sequence coding for the amino acid sequence as indicated in SEQ ID NO: 5.

19. A hypervariable region peptide in VP2 of a passage strain from infectious bursal disease virus TY2 strain having the amino acid sequence as indicated in SEQ ID NO: 5.


SEQUENCE LISTING (13 sequences)

SEQ ID NO: 1 - protein, 507 aa, Infectious bursal disease virus
  misc_feature: VP2 of Infectious Bursal Disease Virus TY2
MTNLADQTQQIVPFIRSLLMPTTGPASIPDDTLEKHTLRSETSTYNLTVGDTGSGLIVFFPGFPGSIVGAHYTLQNNGNYKFDQMLLTAQNLPASYNYCRLVSRSLTVRSSTLPGGVYALNGTVNAVTFQGSLSELTDVSYNGLMSATANINDKIGNVLVGEGVTVLSLPTSYDLGYVRLGDPIPAIGLDPKMVATCDSSDRPRVYTITAADDYQFSSQYQSGGVTITLFSANIDAITSLSVGGELVFQTSVQSLVLDATIYLIGFDGTTVTTRAVAANNGLTAGTDNPIPFNLVFPTHEITQPVTSIKLEIVTSKSGGQAGDQMSWSASGSLAVTIHGGNYPGALRPVTLVAYERVAKGSVVTVAGVSNFELIPNPELAKNLVTEYGRFDPGAMNYTKLILSERDRLGIKTVWPTREYTDFREYFMEVADLNSPLKIAGAFGFKDIIRAIRRIAVPVVSTLFPPAAPLAHAIGEGVDYLLGDEAQAASGTARAASGKARAASGRIR

SEQ ID NO: 2 - DNA, 1523 bp, Infectious bursal disease virus
  misc_feature: Nucleotide Sequence cording for Sequence ID No.1
atgacaaacctggcagatcaaacccaacagattgttccgtttatacggagccttctgatgccaacaaccggaccggcgtccattccggacgacaccctagagaagcacactctcaggtcagagacctcaacttacaatttgactgtgggggacacagggtcagggctaattgtctttttccctggattcccgggctcaattgtgggtgctcactacacactgcagaacaatgggaactacaagttcgatcagatgctcctgactgcccagaacctaccggccagttacaactactgcaggttagtgagtcggagtctcacagtgaggtcaagcacactccctggtggcgtttatgctctaaacggcaccgtaaacgccgtgaccttccaaggaagcctgagtgaactgacagatgttagctacaatgggttgatgtctgcaacggccaacatcaacgacaaaattgggaacgtcctggtaggggaaggggtcaccgtcctcagcctacccacatcatatgatcttgggtatgtgcggcttggtgaccccattcctgctatagggcttgacccaaaaatggtagccacatgtgacagcagtgacaggcccagagtctacaccataaccgcagccgatgattaccaattctcatcacagtaccaatcaggtggggtaacaatcacactgttctcagccaacattgatgccatcacaagcctaagcgttggaggagagctcgtgttccaaaccagcgtccaaagccttgtactggacgccaccatctaccttataggatttgatgggaccacggtaaccaccagagctgtggctgcgaacaatgggctgacggctggcaccgacaatcctattccattcaatcttgtgtttccgacccatgagataacccagccagtcacatccatcaaactggagatagtgacctccaaaagtggtggtcaagctggggatcagatgtcatggtcggcaagtggaagcctagcagtgacaatccatggtggcaactatccaggggccctccgtcctgtcacactagtagcttatgaaagagtggcaaaaggatccgtcgttacagtcgcaggggtgagcaacttcgagctgatcccaaatcctgagctagcaaagaacctggttacagaatacggccgatttgacccaggggcaatgaactacacaaaattgatattgagtgagagggaccgtcttggcatcaagaccgtctggccaacaagggagtacactgactttcgtgagtacttcatggaagtggctgacctcaactctcccctgaagattgcaggagcttttggcttcaaggacataatccgggccataaggaggatagctgtgccggtggtctccacattgtttccacctgccgctcccctagcccatgcaattggggaaggtgtagactacctgctgggcgatgaggctcaagctgcctcaggaactgctcgagccgcgtcaggaaaagcaagagctgcctcaggtcgcataaggca

SEQ ID NO: 3 - protein, 145 aa, Infectious bursal disease virus
  misc_feature: Hyper Variable Region in VP2 of Infectious Bursal Disease Virus TY2; Relevant Residues in SEQ ID 1 from 206 to 350.
YTITAADDYQFSSQYQSGGVTITLFSANIDAITSLSVGGELVFQTSVQSLVLDATIYLIGFDGTTVTTRAVAANNGLTAGTDNPIPFNLVFPTHEITQPVTSIKLEIVTSKSGGQAGDQMSWSASGSLAVTIHGGNYPGALRPVT

SEQ ID NO: 4 - protein, 145 aa, Infectious bursal disease virus
  misc_feature: Hyper Variable Region in VP2 of Infectious Bursal Disease Virus TY2-CEF
YTITAADEYQFSSQYQSGGVTITLFSANIDAITSLSVGGELVFQTSVHSLVLEATIYLIGFDGTTVTTRAVAANNGLTTGTDNPIPFNLVFPTHEITQPVTSIKLEIVTSKSGGQAGDQMSWSASGSLAVTIHGGNYPGALRPVT

SEQ ID NO: 5 - protein, 145 aa, Infectious bursal disease virus
  misc_feature: Hyper Variable Region in VP2 of Infectious Bursal Disease Virus TY2-CEF2
YTITAADNYQFSSQYQSGGVTITLFSANIDAITSLSVGGELVFQTSVQSLVLYATIYLIGFDGTTVTTRAVAANNGLTTGTDNPIPFNLVFPTHEITQPVTSIKLEIVTSRSGGQAGDQMSWSASGSLAVTIHGGNYPGALRPVT

SEQ ID NO: 6 - protein, 145 aa, Infectious bursal disease virus
  misc_feature: Hyper Variable Region in VP2 of Infectious Bursal Disease Virus K
YTITAADDYQFSSQYQPGGVTITLFSANIDAITSLSVGGELVFQTSVHGLVLGATVYFIGFDGTTVITRAVAANNGLTTGTDNLMPFNLVIPTNEITQPITSIKLEVVTSKSGGQAGDQMSWSASGSLAVTIHGGNYPGALRPVT

SEQ ID NO: 7 - protein, 145 aa, Infectious bursal disease virus
  misc_feature: Hyper Variable Region in VP2 of Infectious Bursal Disease Virus 228E
YTITAADDYQFSSQYQSGGVTITLFSANIDAITSLSVGGELVFQTSVQGLILGATIYLIGFDGTTVITRAVAANNGLTAGTDNLMPFNLVIPTNEITQPITSIKLEIVTSKSGGQAGDQMSWSVSGSLAVTTHGGNYPGALRPVT

SEQ ID NO: 8 - protein, 145 aa, Infectious bursal disease virus
  misc_feature: Hyper Variable Region in VP2 of Infectious Bursal Disease Virus 2512
YTITAADDYQFLSQYQPGGVTITLFSANIDAITSLSVGGELVFQTSVQGLVLGATIYLIGFDGTTVITRAVAADNGLTAGTDNLMPFNIVIPTNEITQPITSIKLEIVTSKSGGQAGDQMSWSASGSLAVTIHGGNYPGALRPVT

SEQ ID NO: 9 - protein, 145 aa, Infectious bursal disease virus
  misc_feature: Hyper Variable Region in VP2 of Infectious Bursal Disease Virus Bursine2
YTITAADDYQFSSQYQSGGVTITLFSANIDAITSLSIGGELVFHTSVHGLALDATIYLIGFDGTTVITRAVASDNGLTTGIDNLMPFNLVIPTNEITQPITSIKLEIVTSKSGGQAGDQMSWSASGSLAVTIHGGNYPGALRPVT

SEQ ID NO: 10 - protein, 145 aa, Infectious bursal disease virus
  misc_feature: Hyper Variable Region in VP2 of Infectious Bursal Disease Virus D78
YTITAADDYQFSSQYQPGGVTITLFSANIDAITSLSVGGELVFQTSVHGLVLGATIYLIGFDGTTVITRAVAANNGLTTGTDNLMPFNLVIPTNEITQPITSIKLEIVTSKSGGQAGDQMSWSARGSLAVTIHGGNYPGALRPVT

SEQ ID NO: 11 - protein, 140 aa, Infectious bursal disease virus
  misc_feature: Hyper Variable Region in VP2 of Infectious Bursal Disease Virus V877
ADDYQFSSQYQPGGVTITLFSANIDAITSLSVGGELVFQTSVQGLVLNATIYLVGFDGTTVTTRAVAAGNGLTAGTDNLMPFNLVIPTSEITQPVTSIKLEIVTSKSGGQAGDQMSWLASGNLAVTIHGGNYPGALRPVT

SEQ ID NO: 12 - DNA, 18 bp, Artificial
  primer
cccagagtctacaccata

SEQ ID NO: 13 - DNA, 18 bp, Artificial
  primer
tcctgttgccactctttc